(19) United States
(12) Patent Application Publication
(10) Pub. No.: US 2024/0238236 A1
(43) Pub. Date: Jul. 18, 2024

(54) COMPOSITION FOR REGULATING CARBOHYDRATE METABOLISM, AND METHOD FOR ASSESSING CARBOHYDRATE METABOLISM CONDITIONS OF SUBJECT

(71) Applicants: KAGAMI INC., Ibaraki-shi, Osaka (JP); NATIONAL UNIVERSITY CORPORATION KANAZAWA UNIVERSITY, Kanazawa-shi, Ishikawa (JP)

(72) Inventors: Takashi WADA, Ishikawa (JP); Norihiko SAKAI, Ishikawa (JP); Yasunori IWATA, Ishikawa (JP); Yusuke NAKADE, Ishikawa (JP); Taku KOBAYASHI, Ishikawa (JP); Masashi MITA, Osaka (JP)

(73) Assignees: KAGAMI INC., Ibaraki-shi, Osaka (JP); NATIONAL UNIVERSITY CORPORATION KANAZAWA UNIVERSITY, Kanazawa-shi, Ishikawa (JP)

(21) Appl. No.: 18/561,927

(22) PCT Filed: May 19, 2022
(86) PCT No.: PCT/JP2022/020833

§ 371 Date: Nov. 17, 2023

(30) Foreign Application Priority Data
May 19, 2021 (JP) 2021-084972

(51) Int. Cl. A61K 31/198; A61K 31/519; A61P 3/10

(52) U.S. Cl. CPC A61K 31/198; A61K 31/519; A61P 3/10

(57) ABSTRACT

The present invention provides a composition for regulating carbohydrate metabolism, the composition containing, as an active ingredient, an agent for controlling the amount of D-amino acid in the living body of a subject. Moreover, the present invention provides a method for assessing carbohydrate metabolism conditions of a subject by monitoring the amount of D-amino acid in the living body of the subject.

### FIELD

The present invention relates to a composition for modulating carbohydrate metabolism, and to a method for assessing the state of carbohydrate metabolism of a subject. The invention further relates to a method for modulating carbohydrate metabolism of a subject, and to the use of a modulating agent for the amount of D-amino acids in vivo, for production of a pharmaceutical composition for modulation of carbohydrate metabolism.

### BACKGROUND

Advances in quantitative research of trace D-amino acids and L-amino acids in vivo in organisms such as mammals, due to higher level performance of techniques used to identify and analyze chiral amino acids, have led to elucidation of the presence and function of some D-amino acids which were previously treated as total amino acids (D-amino acids+L-amino acids), or L-amino acids for convenience, due to technical limitations of the prior art.

It has been reported that D-amino acids are present in varying levels in vivo, in tissues, in cells and in body fluids depending on effects such as intake, symbiotic bacteria, metabolism (decomposition and synthesis), transport and excretion (NPLs 1 to 5), that a characteristic chiral amino acid profile is exhibited in diseases such as kidney disease, cardiac disease and diabetes (PTL 1), and that D-amino acids are involved in intestinal immunity (NPL 6) and protect kidney-derived cells (NPL 2). Carbohydrate metabolism in neurons has been reported to be involved in the biosynthesis of D-serine (NPL 7).

Fluctuations in D-alanine, D-proline and D-aspartic acid levels in diabetic blood have been disclosed (PTLs 1 to 2). It has been reported that D-alanine is localized in cells containing insulin in the pancreatic islets of Langerhans, and adrenocorticotropic hormone in the anterior pituitary gland (NPLs 8 to 9). However, the relationship between such localization and diabetes pathology or carbohydrate metabolism still remains to be elucidated. Glucose is the most important energy source in vivo, being taken up from the blood by all cells. The glucose concentration in blood (blood glucose level) is normally maintained within a certain range by hormones from the pancreatic islets of Langerhans, pituitary, adrenal medulla and adrenal cortex, as well as by functioning of the nervous system. When excess glucose is present in the blood after eating, for example, glucose levels are modulated by promoting glycogen synthesis in the liver and muscles primarily by the action of insulin, or by storage as fat in adipose tissue. When glucose levels decrease, insulin antagonistic hormones such as glucagon act to degrade glycogen stored in the liver or muscles into glucose for supply to the blood, thereby keeping glucose levels in the normal range. It is not known how fluctuations in D-amino acid levels affect such carbohydrate metabolism.

### CITATION LIST

### Patent Literature

[PTL 1] International Patent Publication No. WO2013/140785
[PTL 2] Japanese Unexamined Patent Publication No. 2017-207490
[PTL 3] International Patent Publication No. WO2020/196436

### Non Patent Literature

[NPL 1] Y. Miyoshi, R. Konno, J. Sasabe, K. Ueno, Y. Tojo, M. Mita, S. Aiso and K. Hamase, Alteration of intrinsic amounts of D-serine in the mice lacking serine racemase and D-amino acid oxidase, Amino Acids, 43, 1919-1931 (2012). DOI: 10.1007/s00726-012-1398-4
[NPL 2] Y. Nakade, Y. Iwata, K. Furuichi, M. Mita, K. Hamase, R. Konno, T. Miyake, N. Sakai, S. Kitajima, T. Toyama, Y. Shinozaki, A. Sagara, T. Miyagawa, A. Hara, M. Shimizu, Y. Kamikawa, K. Sato, M. Oshima, S. Yoneda-Nakagawa, Y. Yamamura, S. Kaneko, T. Miyamoto, M. Katane, H. Homma, H. Morita, W. Suda, M. Hattori and T. Wada, Gut microbiota-derived D-serine protects against acute kidney injury, JCI Insight, 3 (2018). DOI: 10.1172/jci.insight.97957
[NPL 3] M. Ariyoshi, M. Katane, K. Hamase, Y. Miyoshi, M. Nakane, A. Hoshino, Y. Okawa, Y. Mita, S. Kaimoto, M. Uchida, K. Fukai, K. Ono, S. Tateishi, D. Hato, R. Yamanaka, S. Honda, Y. Fushimura, E. Iwai-Kanai, N. Ishihara, M. Mita, H. Homma and S. Matoba, D-Glutamate is metabolized in the heart mitochondria, Scientific Reports, 7 (2017). DOI: 10.1038/srep43911
[NPL 4] P. Wiriyasermkul, S. Moriyama, Y. Tanaka, P. Kongpracha, N. Nakamae, M. Suzuki, T. Kimura, M. Mita, J. Sasabe and S. Nagamori, D-Serine, an emerging biomarker of kidney diseases, is a hidden substrate of sodium-coupled monocarboxylate transporters, bioRxiv preprint. DOI: 10.1101/2020.08.10.244822
[NPL 5] A. Hesaka, S. Sakai, K. Hamase, T. Ikeda, R. Matsui, M. Mita, M. Horio, Y. Isaka and T. Kimura, D-Serine reflects kidney function and diseases, Scientific Reports, 9 (2019). DOI: 10.1038/s41598-019-41608-0
[NPL 6] J. Sasabe, Y. Miyoshi, S. Rakoff-Nahoum, T. Zhang, M. Mita, B. M. Davis, K. Hamase and M. K. Waldor, Interplay between microbial D-amino acids and host D-amino acid oxidase modifies murine mucosal defence and gut microbiota, Nature Microbiology, 1 (2016). DOI: 10.1038/nmicrobiol.2016.125
[NPL 7] Suzuki, J. Sasabe, Y. Miyoshi, K. Kuwasako, Y. Muto, K. Hamase, M. Matsuoka, N. Imanishi and S. Aiso, Glycolytic flux controls D-serine synthesis through glyceraldehyde-3-phosphate dehydrogenase in astrocytes, Proceedings of the National Academy of Sciences of the United States of America, 112, E2217-2224 (2015). DOI: 10.1073/pnas, 1416117112
[NPL 8] A. Morikawa, K. Hamase, T. Ohgusu, S. Etoh, H. Tanaka, I. Koshiishi, Y. Shoyama and K. Zaitsu, Immunohistochemical localization of D-alanine to ß-cells in rat pancreas, Biochemical and Biophysical Research Communications, 355, 872-876 (2007). DOI: 10.1016/j.bbrc.2007.02.056
[NPL 9] S. Etoh, K. Hamase, A. Morikawa, T. Ohgusu and K. Zaitsu, Enantioselective visualization of D-alanine in rat anterior pituitary gland: localization to ACTH-secreting cells, Analytical and Bioanalytical Chemistry, 393, 217-223 (2009). DOI: 10.1007/s00216-008-2401-5
[NPL 10] Y. Shimizu, C. Ishii, R. Yanobu-Takanashi, K. Nakano, A. Imaike, M. Mita, K. Hamase and T. Okamura, D-Amino acid oxidase deficiency is caused by a large deletion in the Dao gene in LEA rats, Biochimica et Biophysica Acta-Proteins and Proteomics, 1868 (2020). DOI: 10.1016/j.bbapap.2020.140463
[NPL 11] S. Karakawa, Y. Miyoshi, R. Konno, S. Koyanagi, M. Mita, S. Ohdo and K. Hamase, Two-dimensional high-performance liquid chromatographic determination of day-night variation of D-alanine in mammals and factors controlling the circadian changes, Analytical and Bioanalytical Chemistry, 405, 8083-8091 (2013). DOI: 10.1007/s00216-013-7071-2
[NPL 12] Y. Miyoshi, H. Kenji, Y. Tojo, M. Mita, R. Konno and K. Zaitsu, Determination of D-serine and D-alanine in the tissues and physiological fluids of mice with various D-amino-acid oxidase activities using two-dimensional high-performance liquid chromatography with fluorescence detection, Journal of Chromatography B, 877, 2506-2512 (2009). DOI: 10.1016/j.jchromb.2009.06.028
[NPL 13] H. HAN, Y. Miyoshi, R. Koga, M. MITA, R. Konno, K. Hamase, Changes in D-aspartic acid and D-glutamic acid levels in the tissues and physiological fluids of mice with various D-aspartate oxidase activities, Journal of Pharmaceutical and Biomedical Analysis, 116, 47-52 (2015). DOI: 10.1016/j.jpba.2015.05.013

### SUMMARY

### Technical Problem

In conditions such as diabetes where carbohydrate metabolism or transport in vivo, in tissues, in cells, in organs or in body fluids is abnormal, body glucose levels exhibit abnormal values which can lead to a variety of related diseases, and therefore methods for modulating glucose levels are highly desired.

### Solution to Problem

The present inventors have discovered a surprising phenomenon in which knock-out mice (DAO−/− mice) lacking the gene encoding D-amino acid oxidase (DAO) which is thought to be involved in metabolism of D-amino acid levels in vivo, in tissues, in cells, in organs and in body fluids, exhibit decreased glucose levels in the blood (blood glucose levels). Since it is known that D-amino acid levels are increased in DAO−/− mice and rats (NPL 1 and NPLs 10 to 12), the present inventors conceived of the idea of artificially altering the levels of D-amino acids in vivo to modulate carbohydrate metabolism and alter the levels of glucose or insulin in the blood. As a result of further ardent research on this possibility, a method was found for modulating carbohydrate metabolism, and specifically glucose levels in blood, by administration of D-amino acids or inhibition of metabolic enzymes, which can increase or decrease D-amino acid levels in vivo, and the present invention was completed. Specifically, the present invention encompasses the following.

[1] A composition for modulating carbohydrate metabolism, comprising a modulating agent for the amount of D-amino acids in a subject in vivo, as an active ingredient.

[2] The composition according to aspect [1] above, wherein the modulating of carbohydrate metabolism is modulating of the glucose level in blood.

[3] The composition according to aspect [1] or [2] above, wherein the modulating agent is a D-amino acid, or modified form or derivative thereof.

[4] The composition according to aspect [3] above, wherein the D-amino acid is selected from the group consisting of D-alanine, D-serine and D-leucine.

[5] The composition according to aspect [3] or [4] above, wherein the modulating of carbohydrate metabolism is improvement in carbohydrate metabolism.

[6] The composition according to aspect [1] or [2] above, wherein the modulating agent is an agent that regulates the activity of a protein associated with absorption, transport, distribution, metabolism or excretion of a D-amino acid.

[7] The composition according to aspect [6] above, wherein the metabolism is decomposition and/or synthesis.

[8] The composition according to aspect [6] or [7] above, wherein the agent that regulates the activity of the protein is a modulating agent for gene expression of the protein.

[9] The composition according to any one of aspects [6] to [8] above, wherein the modulating agent is selected from the group consisting of antisense RNA or DNA molecules, RNAi-inducible nucleic acids, microRNA (miRNA), ribozymes, genome editing nucleic acids and their expression vectors, low molecular compounds, aptamers, antibodies, antibody fragments, and combinations of the foregoing.

[10] The composition according to any one of aspects [6] to [9] above, wherein the protein is selected from the group consisting of D-amino acid oxidase, D-aspartate oxidase and serine isomerase.

[11] The composition according to any one of aspects [1] to above, wherein the modulating agent is Risperidone.

[12] The composition according to any one of aspects [1] to above, wherein the carbohydrate metabolism is carbohydrate metabolism by secretion and/or action of insulin and/or glucagon.

[13] The composition according to any one of aspects [1] to above, wherein the carbohydrate metabolism is carbohydrate metabolism represented by insulin resistance or sensitivity, or glucose tolerance.

[14] The composition according to aspect [1] or [2] above, wherein the source of the D-amino acid is symbiotic bacteria.

[15] The composition according to aspect [1] or [2] above, wherein the subject is a subject with a carbohydrate metabolism disorder.

[16] The composition according to aspect above, wherein the carbohydrate metabolism disorder is diabetes.

[17] The composition according to aspect [1] or [2] above, wherein the subject is a subject with a diabetic complication.

[18] The composition according to aspect above, wherein the diabetic complication is selected from the group consisting of retinopathy, nephropathy, neuropathy and arteriosclerosis.

[19] The composition according to any one of aspects [1] to above, which is a drug.

[20] The composition according to any one of aspects [1] to above, which is a food.

[21] The composition according to aspect above, wherein the food is a health functional food or dietary supplement.

[22] The composition according to aspect above, wherein the health functional food is a specified health food or nutritional function food.

[23] The composition according to any one of aspects [1] to above, which improves blood pressure.

[24] A composition for modulating blood pressure, comprising a modulating agent for the amount of D-amino acids a subject in vivo, as an active ingredient.

[25] The composition according to aspect above, wherein the modulating of blood pressure is lowering of blood pressure.

[26] A method for assessing the state of carbohydrate metabolism of a subject by monitoring the level of D-amino acids in the subject in vivo.

[27] The method according to aspect above, wherein the subject is a subject with a carbohydrate metabolism disorder.

[28] The method according to aspect above, wherein the carbohydrate metabolism disorder is diabetes.

[29] A method for modulating carbohydrate metabolism of a subject, wherein the method comprises:
administering a modulating agent for the amount of D-amino acids in vivo to a subject in need thereof.

[30] Use of a modulating agent for the amount of D-amino acids in vivo, for production of a pharmaceutical composition for modulation of carbohydrate metabolism.

### Advantageous Effects of Invention

According to the invention it is possible to modulate D-amino acid levels in vivo in order to modulate carbohydrate metabolism and especially blood glucose level, and to thereby prevent, treat or evaluate diseases with abnormal carbohydrate metabolism (such as diabetes).

### BRIEF DESCRIPTION OF DRAWINGS

FIG. 1 is a graph showing fasting blood insulin levels in D-amino acid- and carbohydrate metabolism-modified mice (type 1 diabetes model).

FIG. 2 is a set of graphs showing blood glucose levels under insulin tolerance testing in D-amino acid- and carbohydrate metabolism-modified mice (type 1 diabetes model).

FIG. 3 is a graph showing blood glucose levels under glucose tolerance testing in D-amino acid- and carbohydrate metabolism-modified mice (type 1 diabetes model).

FIG. 4 is a graph showing casual blood glucose levels in D-amino acid- and carbohydrate metabolism-modified mice (type 1 diabetes model).

FIG. 5 is a graph showing fasting blood glucose levels in D-amino acid- and carbohydrate metabolism-modified mice (type 1 diabetes model).

FIG. 6 is a graph showing insulin resistance indexes in D-amino acid- and carbohydrate metabolism-modified mice (type 1 diabetes model).

FIG. 7 is a pair of graphs showing systolic pressure and diastolic pressure in D-amino acid- and carbohydrate metabolism-modified mice (type 1 diabetes model).

FIG. 8 is a pair of graphs showing blood glucose levels under insulin tolerance testing in carbohydrate metabolism-modified mice (type 1 diabetes model) administered a D-amino acid metabolic enzyme inhibitor.

FIG. 9 is graph showing blood glucose levels under glucose tolerance testing in carbohydrate metabolism-modified mice (type 1 diabetes model) administered a D-amino acid metabolic enzyme inhibitor.

FIG. 10 is a graph showing casual blood glucose levels in carbohydrate metabolism-modified mice (type 1 diabetes model) administered a D-amino acid metabolic enzyme inhibitor.

FIG. 11 is a graph showing fasting blood glucose levels in carbohydrate metabolism-modified mice (type 1 diabetes model) administered a D-amino acid metabolic enzyme inhibitor.

FIG. 12 is a pair of graphs showing blood glucose levels under insulin tolerance testing in carbohydrate metabolism-modified mice (type 1 diabetes model) administered a D-amino acid.

FIG. 13 is a graph showing fasting blood glucose levels in carbohydrate metabolism-modified mice (type 1 diabetes model) administered a D-amino acid.

FIG. 14 is a graph showing fasting blood glucose levels in carbohydrate metabolism-modified mice (type 1 diabetes model) administered a D-amino acid.

FIG. 15 is a pair of graphs showing systolic pressure and diastolic pressure in carbohydrate metabolism-modified mice (type 1 diabetes model) administered a D-amino acid.

FIG. 16 is a graph showing fasting blood insulin levels in carbohydrate metabolism-modified mice (type 1 diabetes model) administered a D-amino acid-free diet or a D-amino acid.

FIG. 17 is a graph showing non-fasting blood glucose levels in carbohydrate metabolism-modified mice (type 1 diabetes model) administered a D-amino acid-free diet or a D-amino acid.

FIG. 18 is a graph showing fasting blood glucose levels in carbohydrate metabolism-modified mice (type 1 diabetes model) administered a D-amino acid-free diet or a D-amino acid.

FIG. 19 is a pair of graphs showing insulin and glucagon levels in pancreatic islets of Langerhans, in carbohydrate metabolism-modified mice (type 1 diabetes model) administered a D-amino acid-free diet or a D-amino acid.

FIG. 20 is a graph showing non-fasting blood glucose levels in carbohydrate metabolism-modified mice (type 2 diabetes model).

### DESCRIPTION OF EMBODIMENTS

Embodiments for carrying out the invention will be described in detail below, with the understanding that the technical scope of the invention is not limited only to these embodiments. The prior art documents cited throughout the present specification are incorporated herein by reference.

One embodiment of the invention provides a composition for modulating carbohydrate metabolism, and specifically blood glucose level, comprising a modulating agent for the amount of D-amino acids in a subject in vivo, as an active ingredient. The present invention was completed after finding that carbohydrate metabolism, and specifically blood glucose level, can be modulated by increasing or decreasing D-amino acid levels in vivo.

As used herein, the phrase “modulate D-amino acid levels in vivo” means intentionally increasing or decreasing the level of a D-amino acid in vivo (for example, in cells, tissue, organs or body fluids). When a specific target level and concentration exist, evaluation may be made by appropriate monitoring of the D-amino acid in a biological sample.

The term “modulating agent for the amount of D-amino acids” (also, “D-amino acid level modulating agent”) as used herein refers to an agent that can increase or decrease D-amino acid levels in vivo (including cells, tissues, organs or body fluids) of a subject by its application (such as administration). The phrase “modulate the amount of D-amino acids in cells” as used herein means to increase or decrease the levels of D-amino acids in cells to modulate the levels of the D-amino acids to a desired range, by application of a D-amino acid level modulating agent. The phrase “modulate the amount of D-amino acids in tissues” as used herein means to increase or decrease the levels of D-amino acids in a tissue (such as kidney tubules or glomeruli) to modulate the levels of the D-amino acids to a desired range, by application of a D-amino acid level modulating agent. The phrase “modulate the amount of D-amino acids in organs” as used herein means to increase or decrease the levels of D-amino acid in an organ (such as the kidneys or heart) to modulate the levels of the D-amino acids to a desired range, by application of a D-amino acid level modulating agent. The phrase “modulate the amount of D-amino acids in body fluids” as used herein means to increase or decrease the levels of D-amino acids in a body fluid (such as blood or urine) to modulate the levels of the D-amino acids to a desired range, by application of a D-amino acid level modulating agent.

The term “D-amino acids” is used herein to include “D-isomers” of amino acids that are constituents of proteins which are stereoisomers of “L-isomers” of amino acids that are constituents of proteins, as well as glycine which has no stereoisomer, and specifically they include glycine. D-alanine. D-histidine. D-isoleucine. D-allo-isoleucine. D-leucine. D-lysine. D-methionine. D-phenylalanine. D-threonine. D-allo-threonine. D-tryptophan. D-valine. D-arginine. D-cysteine. D-glutamine. D-proline. D-tyrosine. D-aspartic acid. D-asparagine, D-glutamic acid and D-serine. Since D-cysteine in a biological sample is oxidized ex vivo and converted to D-cystine, one embodiment of the invention allows measurement of D-cystine instead of D-cysteine to determine the level of D-cysteine in the biological sample.

As used herein, a “biological sample” is a sample derived from an organism, and it may be blood, blood plasma, serum, saliva, urine, ascites fluid, amnionic fluid, lymph, semen, spinal fluid, nasal discharge, sweat, milk or tears, cells and tissues, although blood, blood plasma or serum is preferred as the biological sample for the invention.

The level of a D-amino acid and/or L-amino acid can be determined by any method, such as chiral column chromatography, enzymatic methods, and even immunological method using a monoclonal antibody that identify optical isomers of amino acids. Measurement of a D-amino acid and/or L-amino acid level in a sample herein may be carried out using any method well known to those skilled in the art. Examples include chromatographic and enzyme methods (Y. Nagata et al., Clinical Science, 73 (1987), 105. Analytical Biochemistry, 150 (1985), 238., A. D′Aniello et al., Comparative Biochemistry and Physiology Part B, 66 (1980), 319. Journal of Neurochemistry, 29 (1977), 1053., A. Berneman et al., Journal of Microbial & Biochemical 20) Technology, 2 (2010), 139., W. G. Gutheil et al., Analytical Biochemistry, 287 (2000), 196., G. Molla et al., Methods in Molecular Biology, 794 (2012), 273., T. I to et al., Analytical Biochemistry, 371 (2007), 167.), antibody methods (T. Ohgusu et al., Analytical Biochemistry, 357 (2006), 15), gas chromatography (GC) (H. Hasegawa et al., Journal of Mass Spectrometry, 46 (2011), 502., M. C. Waldhier et al., Analytical and Bioanalytical Chemistry, 394 (2009), 695., A. Hashimoto. T. Nishikawa et al., FEBS Letters, 296 (1992), 33., H. Bruckner and A. Schieber. Biomedical Chromatography, 15 (2001), 166., M. Junge et al., Chirality, 19 (2007), 228., M. C. Waldhier et al., Journal of Chromatography A, 1218 (2011), 4537), capillary electrophoresis methods (CE) (H. Miao et al., Analytical Chemistry, 77 (2005), 7190., D. L. Kirschner et al., Analytical Chemistry, 79 (2007), 736., F. Kitagawa. K. Otsuka. Journal of Chromatography B, 879 (2011), 3078., G. Thorsen and J. Bergquist. Journal of Chromatography B, 745 (2000), 389), and high-performance liquid chromatography (HPLC) (N. Nimura and T. Kinoshita. Journal of Chromatography, 352 (1986), 169., A. Hashimoto et al., Journal of Chromatography, 582 (1992), 41., H. Bruckner et al., Journal of Chromatography A, 666 (1994), 259., N. Nimura et al., Analytical Biochemistry, 315 (2003), 262., C. Muller et al., Journal of Chromatography A, 1324 (2014), 109., S. Einarsson et al., Analytical Chemistry, 59 (1987), 1191., E. Okuma and H. Abe. Journal of Chromatography B, 660 (1994), 243., Y. Gogami et al., Journal of Chromatography B, 879 (2011), 3259., Y. Nagata et al., Journal of Chromatography, 575 (1992), 147., S. A. Fuchs et al., Clinical Chemistry, 54 (2008), 1443., D. Gordes et al., Amino Acids, 40 (2011), 553., D. Jin et al., Analytical Biochemistry, 269 (1999), 124., J. Z. Min et al., Journal of Chromatography B, 879 (2011), 3220., T. Sakamoto et al., Analytical and Bioanalytical Chemistry, 408 (2016).

517., W. F. Visser et al., Journal of Chromatography A, 1218 (2011), 7130., Y. Xing et al., Analytical and Bioanalytical Chemistry, 408 (2016), 141., K. Imai et al., Biomedical Chromatography, 9 (1995), 106., T. Fukushima et al., Biomedical Chromatography, 9 (1995), 10., R. J. Reischl et al., Journal of Chromatography A, 1218 (2011), 8379., R. J. Reischl and W. Lindner. Journal of Chromatography A, 1269 (2012), 262., S. Karakawa et al., Journal of Pharmaceutical and Biomedical Analysis, 115 (2015), 123., Hamase K. et al., Chromatography 39 (2018) 147-152).

The separative analysis system for optical isomers according to the invention may be a combination of multiple separative analysis methods. More specifically, a D-amino acid and/or L-amino acid level in a sample can be measured using a method for analyzing optical isomers, comprising the following steps: passing a sample containing a component with optical isomers through a first column filler as a stationary phase, together with a first liquid as a mobile phase, to separate the components in the sample; retaining each of the components from the sample separately in a multi loop unit; feeding each of the components from the sample separately retained in the multi loop unit, together with a second liquid as a mobile phase, through a flow channel to a second column filler having an optically active center, as a stationary phase, to separate the optical isomers of each component of the sample; and detecting the optical isomers of each component from the sample (Japanese Patent No. 4291628). In HPLC analysis. D- and L-amino acids may be pre-derivatized with a fluorescent reagent such as o-phthalaldehyde (OPA) or 4-fluoro-7-nitro-2,1,3-benzoxadiazole (NBD-F), or diastereomerized using an agent such as N-tert-butyloxycarbonyl-L-cysteine (Boc-L-Cys) (Hamase. K. and Zaitsu. K., Bunseki Kagaku. Vol. 53, 677-690(2004)). Alternatively, the D-amino acids or L-amino acids may be measured by an immunological method using a monoclonal antibody that identifies optical isomers of amino acids, such as a monoclonal antibody that specifically binds to a D-amino acid or L-amino acid. On the other hand, when the total of the D-isomers and L-isomers are to be used as an indicator, it is not necessary to analyze D-isomers and L-isomers separately, but the amino acids can be analyzed without distinguishing between D-isomers and L-isomers. In such cases as well, separation and quantitation may be carried out using an enzyme method, antibody method, GC, CE or HPLC, etc.

As used herein, “modulating of carbohydrate metabolism” means using a prescribed method to restore a state of balance or a proper state against disorder of catabolismor assimilation of nutrients (such as carbohydrates) or an excess or deficiency thereof in a subject. When there is a desired effect to achieve by modulating of carbohydrate metabolism, it may be evaluated by appropriately monitoring insulin level, glucose level or a metabolism-related marker in a biological sample, or by conducting a glucose tolerance test or insulin tolerance test. According to one embodiment, the “modulating of carbohydrate metabolism” to be achieved by the invention may be improvement in carbohydrate metabolism, and it is preferably modulation of blood glucose level (such as lowering of blood glucose level).

It is known that increasing glucose level in the blood increases the blood osmotic pressure and therefore promotes reabsorption of water in the kidneys and release of water from cells, thereby increasing blood volume and body fluid volume and consequently increasing blood pressure. According to one embodiment, therefore, the “modulating of carbohydrate metabolism” to be achieved by the invention may include modulation of blood pressure, such as improvement in blood pressure (such as lowering of hypertension). According to another embodiment, the invention provides a composition for modulating blood pressure comprising a modulating agent for the amount of D-amino acids in a subject in vivo, as an active ingredient. The phrase “modulating blood pressure” as used herein includes the concept of bringing blood pressure (peak blood pressure (systolic pressure) and/or base blood pressure (diastolic pressure) that has deviated from the normal range (target blood pressure value) to within or near the normal range (target blood pressure value), or approaching the range, and for example, it includes lowering blood pressure generally judged as hypertension.

Abnormal carbohydrate metabolism associated with changes in insulin sensitivity or resistance results in excess or deficient insulin secretion from the pancreas. According to one embodiment, therefore, the “modulating of carbohydrate metabolism” to be achieved by the invention may be modulation of insulin secretion, such as improvement in insulin secretion, and preferably improvement in hyperinsulinemia.

Throughout the present specification, the amounts of biomolecules such as D-amino acids, glucose, insulin and proteins, or drugs, may be expressed in any physical quantity that can be measured, which includes not only the simple mass, weight and amount of substance (mol), but also the mass, weight or amount of substance (mol) per tissue, cell, organ or molecular units or per volume or weight, and the mass, weight, amount of substance (mol), concentration, specific gravity or density in a fluid such as blood or urine.

The D-amino acid level modulating agent to be used for the invention may be a drug or food that can increase or decrease D-amino acid levels in tissues, cells, organs or body fluids by external administration of the D-amino acids, or by addition or removal of the D-amino acids to or from foods. For example, drinking a water-soluble solution containing D-amino acids can increase the D-amino acid concentration in blood or tissues (NPL 1), while ingesting foods with the D-amino acids removed can decrease D-amino acid concentrations in blood. The D-amino acids to be used may also contain D-amino acid modified forms or derivatives or pharmaceutically acceptable salts thereof, so long as they can increase or decrease D-amino acid levels in vivo, and may also include pharmacologically acceptable carriers, diluents or excipients, or may be prepared as prodrugs. A metabolism improver or hypoglycemic agent may also be included in addition to the D-amino acid level modulating agent. A drug to be used for the invention may be formulated in a dosage form selected to be suitable for the route of administration. The dosage form may be designed as a tablet, capsule, liquid drug, powdered drug, granules or a chewable agent for use in oral administration, or as an injection, powdered drug or infusion preparation for parenteral administration. These formulations may also include various types of adjuvants such as carriers or other auxiliary agents that are used in drugs, including stabilizers, antiseptic agents, soothing agents, flavorings, taste correctives, aromatics, emulsifiers, fillers and pH adjustors, in ranges that do not interfere with the effect of the invention. The optical purity of the drug and its D-amino acid starting materials is preferably 50% or greater and more preferably 90% or greater, but the optical purity is not restricted and may be selected as desired within a range that exhibits an effect.

According to one embodiment, the D-amino acid level modulating agent to be used for the invention may be selected from the group consisting of D-alanine, D-serine and D-leucine, and modified forms and derivatives thereof.

By applying the invention it is possible to change the amounts of D-amino acids in vivo utilizing different physiological mechanisms, and to modulate carbohydrate metabolism as a result. According to one aspect, the levels of D-amino acids in vivo can be modulated by modulating (promoting or inhibiting, etc.) expression and/or activity (action, inhibition or stimulation) of proteins related to absorption, transport, distribution, metabolism (synthesis and/or decomposition), excretion or action of D-amino acids, or of D-amino acid transporters or receptors.

The modulating agent for the amount of D-amino acid to be used for the invention may therefore be one that directly or indirectly promotes gene expression of a protein related to absorption, transport, distribution, metabolism or excretion of a D-amino acid, and for example, it may be the protein or a vector that expresses it, or it may be a factor that promotes activity upstream in a cascade that promotes expression of the protein, or a vector that expresses the factor.

The modulating agent for the amount of D-amino acids to be used for the invention may also be, for example, one that directly or indirectly inhibits gene expression of a protein related to absorption, transport, distribution, metabolism or excretion of a D-amino acid, such as one selected from among low molecular compounds, aptamers, antibodies or antibody fragments, or antisense RNA or DNA molecules, RNAi-inducible nucleic acid, microRNA (miRNA), ribozymes or genome editing nucleic acids, as well as expression vectors thereof.

Throughout the present specification, a protein related to absorption, transport, distribution, metabolism (synthesis and/or decomposition), excretion or action of a D-amino acid, may be an enzyme, for example, such as D-amino acid oxidase (DAO), D-aspartate oxidase (DDO), serine isomerase (SRR) or DPP-4. For example, a DAO inhibitor (such as Risperidone, chlorpromazine or sodium benzoate) inhibits oxidation of D-amino acids and is able to increase D-amino acid levels at action sites, and it can therefore be used as a modulating agent for D-amino acid levels according to the invention.

Since a D-amino acid transporter can increase or decrease the amount of D-amino acids at the site of pre-transport or transport destination thereof, an agent that directly or indirectly acts on D-amino acid transporters may also be used for the invention.

NPL 4 discloses that agonist/inhibitor D-amino acid transporter proteins such as the SMCT family or ASCT family which are expressed in the brain and kidneys affect local levels of D-amino acids. These transporters are affected by coordination or competition via cotransport substances (such as sodium ions) or scaffolds, and therefore D-amino acid transport activity can also be modulated by sodium/glucose symporter (SGLT2) inhibitors, for example. Therefore, without being limitative, proteins related to absorption, transport, distribution, metabolism (synthesis and/or decomposition), excretion or action of D-amino acids may also be proteins of the SMCT family or ASCT family.

PTL 3 discloses that angiotensin 2 receptor blocker (ARB) alters D-amino acid levels in the blood. Therefore, without being limitative, a protein related to absorption, transport, distribution, metabolism (synthesis and/or decomposition), excretion or action of D-amino acids may be an angiotensin 2 receptor.

As used herein, “aptamer” refers to a synthetic DNA or RNA molecule or peptide molecule that has the ability to specifically bind to a target substance, and it can be chemically synthesized rapidly in vitro. An aptamer used for the invention binds to a protein related to absorption, transport, distribution, metabolism or excretion of D-amino acids, thereby inhibiting its activity. The aptamer to be used for the invention can be obtained, for example, by selection by repetitive in vitro binding to various molecular targets such as small molecules, proteins and nucleic acids, using the SELEX method (see Tuerk C., Gold L., Science, 1990, 249(4968), 505-510; Ellington A D, Szostak J W., Nature, 1990, 346(6287):818-822; U.S. Pat. Nos. 6,867,289; 5,567,588; and 6,699,843).

As used herein, “antibody fragment” refers to a part of a full length antibody that maintains the activity of binding with antigen, and the concept generally includes the antigen-binding domain or variable domain. Examples of antibody fragments include F(ab′)2, Fab′, Fab and Fv antibody fragments (including scFv antibody fragments). The concept of antibody fragment also includes a fragment that is treated with a protease enzyme, often being reduced. The antibody or antibody fragment used for the invention may be any antibody such as a human-derived antibody, mouse-derived antibody, rat-derived antibody, rabbit-derived antibody, Camelidae (such as llama)-derived antibody or goat-derived antibody, and it may also be a polyclonal or monoclonal antibody, or a complete or shortened antibody (for example, a F(ab′)2, Fab′, Fab or Fv fragment), or a chimeric antibody, humanized antibody or fully human antibody.

As used herein, an “antisense RNA or DNA molecule” is a molecule having a nucleotide sequence complementary to functional RNA (sense RNA), such as messenger RNA (mRNA), and that forms a double strand with the sense RNA, having the function of inhibiting synthesis of the protein that is normally carried out by the sense RNA. According to the invention, an antisense oligonucleotide containing an antisense RNA or DNA molecule binds with mRNA of a protein related to absorption, transport, distribution, metabolism or excretion of D-amino acids, thereby inhibiting its translation to protein. This can reduce the expression level of the protein related to absorption, transport, distribution, metabolism or excretion of D-amino acids, thereby inhibiting its activity. The method of synthesizing the antisense RNA or DNA molecule for the invention may be any method known in the technical field.

As used herein, “RNAi-inducible nucleic acid” refers to a polynucleotide that is capable of inducing RNA interference (RNAi) by being introduced into cells, and it may usually be RNA or DNA, or a chimeric molecule of RNA and DNA, comprising 19 to 30 nucleotides, preferably 19 to 25 nucleotides and more preferably 19 to 23 nucleotides, optionally with desired modification. The RNAi may be produced on the mRNA, or on transcribed RNA just before processing, i.e. RNA having a nucleotide sequence including the exon, intron, 3′-untranslated region and 5′-untranslated region. The RNAi method that may be used for the invention may be induction of RNAi by a method such as (1) directly introducing short double-stranded RNA (siRNA) into cells, (2) incorporating short hairpin RNA (shRNA) into expression vectors and introducing the vectors into cells, or (3) creating a vector that expresses siRNA by inserting short double-stranded DNA corresponding to the siRNA, between promoters in a vector having two promoters running in opposite directions, and introducing the vector into cells. The RNAi-inducible nucleic acid may include siRNA, shRNA or miRNA capable of cleaving D-serine transporter protein RNA or suppressing its function, and such RNAi nucleic acid may be directly introduced using liposomes or the like, or it may be introduced using an expression vector that induces the RNAi nucleic acid.

According to one embodiment, the RNAi-inducible nucleic acid for a protein related to absorption, transport, distribution, metabolism or excretion of D-amino acids to be used for the invention may be nucleic acid that exhibits a biological effect of inhibiting or significantly suppressing expression of the protein related to absorption, transport, distribution, metabolism or excretion of D-amino acids, and it can be synthesized by a person skilled in the art by referring to the nucleotide sequence of the protein. For example, it may be chemically synthesized using a DNA (/RNA) automatic synthesizer utilizing DNA synthesis technology such as the solid phase phosphoramidite method, or it may be synthesized by consignment to an siRNA-related contracted synthesis company (such as Life Technologies). According to an embodiment, the siRNA to be used for the invention may be one derived from short-hairpin-type double stranded RNA (shRNA) as the precursor, via processing with a dicer, which may be an intracellular RNase.

As used herein, “microRNA (miRNA)” is a single-stranded RNA molecule with a length of 21 to 25 bases, which contributes to modulation of post-transcriptional expression of genes in eukaryotes. Such miRNA generally recognizes 3′UTR in mRNA, inhibiting translation of target mRNA and inhibiting protein production. Thus, miRNA that can directly and/or indirectly decrease expression levels of a D-serine transporter protein is also within the scope of the present invention.

As used herein, “ribozyme” is a general term for enzymatic RNA molecules that can catalyze specific cleavage of RNA. Ribozymes include large ones of 400 or more nucleotides such as MI RNA, which are included in group I introns or RNase P, but some have active domains of about 40 nucleotides, known as hammerhead types or hairpin types (see Koizumi, M. and Ohtsuka, E., Tanpakushitsu, Kakusan, Kouso, 1990, 35, 2191, for example).

For example, the self-cleaving domain of hammerhead ribozyme cleaves the 3′-end of C15 in the sequence G13U14C15, with formation of a base pair between U14 and A9 being considered important for activity, and potential cleavage at A15 or U15 instead of C15 (see Koizumi, M. et al., FEBS Lett, 1988, 228, 228, for example). If a ribozyme is designed with the substrate binding site being complementary to the RNA sequence near the target site, then it is possible to obtain a restriction enzyme RNA-cleaving ribozyme that recognizes the sequence UC, UU or UA in target RNA, and this can be produced by a person skilled in the art, with reference to the following publications: Koizumi, M. et al., FEBS Lett, 1988, 239, 285; Koizumi, M and Ohtsuka, E. Tanpakushitsu, Kakusan, Kouso, 1990, 35, 2191; and Koizumi, M. et al., Nucl. Acids Res., 1989, 17, 7059).

A hairpin ribozyme may also be used for the invention. This type of ribozyme is found, for example, on the minus strand of satellite RNA of tobacco ringspot virus (Buzayan, J M., Nature, 1986, 323, 349). It has been demonstrated that a target-specific RNA-cleaving ribozyme can be created from a hairpin ribozyme as well (see Kikuchi, Y. & Sasaki, N., Nucl. Acids. Res., 1991, 19, 6751; and Kikuchi, Y., Kagaku to Seibutsu, 1992, 30, 112, for example). By using a ribozyme to specifically cleave the transcription product of a gene coding for a D-serine transporter protein, it is possible to inhibit expression of the D-serine transporter protein.

As used herein, “genome editing nucleic acid” refers to a nucleic acid used for editing of a desired gene in a system utilizing a nuclease that is used for gene targeting. Nucleases used for gene targeting include known nucleases, and also novel nucleases to be used for future gene targeting. For example, known nucleases include CRISPR/Cas9 (Ran, F. A., et al., Cell, 2013, 154, 1380-1389), TALEN (Mahfouz, M., et al., PNAS, 2011, 108, 2623-2628) and ZFN (Urnov, F., et al., Nature, 2005, 435, 646-651).

According to one aspect, utilizing the fact that symbiotic bacteria such as enterobacteria are a source of D-amino acids, the microbiome or growth environment may be altered by means such as administration of antibiotics, intestinal regulators or oligosaccharides, or using probiotics, microbial transplant, fecal transplant or improvement of dysbiosis, thus making it possible to increase or decrease D-amino acid levels in vivo. Without being limitative, one example of probiotics is intake of yogurt containing 1073R-1 lactic acid bacteria, which is known to increase D-serine and decrease D-lysine in the stool, and such lactic acid bacteria may also be used as a modulating agent for D-amino acid levels according to the invention. However, foods known to contain D-amino acids, including microbe fermentation products such as rice vinegar, yogurt, cheese and natto, and bacteria or bacterial extracts, also contain many other active ingredient candidates in addition to D-amino acids, and therefore when these are used as a modulating agent for the amount of D-amino acids for the invention it is essential to use them in amounts that can ensure increase or decrease in D-amino acid levels at the site of action. L-amino acids exhibit various physiological activity different from D-amino acids, and thus foods without established standards (optical purity or amounts) as active ingredients that increase or decrease D-amino acid levels are not included within the scope of the composition of the invention.

A drug or food that can increase or decrease D-amino acid levels in tissues, cells, organs or body fluids, regardless of the mechanism, can be used as means for modulating D-amino acid levels in vivo according to the invention.

Throughout the present specification, the term “drug” is used to include drugs and quasi drugs.

Throughout the present specification, the term “food” means food in general, but in addition to common foods including health foods, it also includes health functional foods such as specified health foods and nutritional function foods, as well as dietary supplements (supplements and nutritional supplements), feeds and food additives.

The method of administration of a composition of the invention may be as a dosage form for local administration (skin, inhalation, enema, eye drop, ear drop, nasal or intravaginal), enteral administration or parenteral administration (intravenous, transarterial, transdermal or intramuscular injection), with enteral administration being preferred. Enteral administration includes oral administration, tube administration and enema administration. Tube administration includes administration through a nasal stomach tube or gastric fistula, or duodenal fistula.

Enema administration includes administration using a suppository or enema. In any case there is no particular restriction on the dosage form of the drug, which may be liquid or solid, and produced by common technical knowledge to those skilled in the art. The specific method of administration is also not particularly restricted, and it may be suitable administration according to common technical knowledge to those skilled in the art.

The subject to which the invention is applied may be a healthy person or a patient diagnosed with or suspected of having abnormal carbohydrate metabolism or nutrition. Diseases with abnormal carbohydrate metabolism or nutrition include diabetes and carbohydrate metabolism disorders (such as glycogen storage disease or galactosemia), or hypoglycemia. Diabetes is a disease in which hyperglycemia is exhibited due to deficient insulin activity (insulin secretion disorder or increased insulin resistance), and it is classified according to the cause, as type 1 diabetes or type 2 diabetes, or due to a specific mechanism or condition (genetic abnormality, pancreatic exocrine disease, endocrine disease, liver disease, drug- or chemical substance-induced, infectious, immunological, or other genetic syndrome), or gestational diabetes. Type 1 diabetes has onset between childhood and adolescence and is assessed by polyuria, mouth dryness, high liquid intake, weight loss, coma (diabetic ketoacidosis), increased blood glucose. HbAlc increase, glycohemoglobin increase, glycosuria increase, urine C peptide (CPR) decrease and GAD antibody increase, and treated primarily by insulin injection. Type 2 diabetes has onset in middle age or older and is assessed by obesity, family history, postprandial hyperglycemia. HbAlc increase, glycohemoglobin increase, polyuria, mouth dryness, high liquid intake, weight loss, fasting blood glucose increase and casual blood glucose increase, and for non-insulin-dependent conditions, is treated by food and exercise therapy or hypoglycemic agents (such as α-glucosidase inhibitors, biguanide agents, thiazolidine inducers, rapid-acting insulin secretagogues, glinide drugs. DPP-4 inhibitors, sulfonylurea agents. SU agents and SGLT2 inhibitors). GLP-1 receptor agonists or insulin injection. For insulin-dependent conditions, treatment is by insulin injection to supplement basal and additional insulin secretion, and food or exercise therapy. Diabetes may also present with diabetic complications such as retinopathy, nephropathy, neuropathy, ischemic heart disease, cerebral infarction.

30) atherosclerosis, ulceration/gangrene (especially foot lesions) and coma, which are also treatment targets of the invention. Animals with genetic modification- or drug-induced carbohydrate metabolism disorders, or biologically-derived or cultured cells, tissues or organoids, can be considered a model representing one state of the human carbohydrate metabolic system, and may be used as subjects.

According to one embodiment of the invention, since increasing or decreasing of D-amino acid levels in a tissue, cells, organ or body fluid of a subject affects carbohydrate metabolism, D-amino acid levels in vivo may be measured as an index of the effect of treatment for a condition of carbohydrate metabolism disorder by a drug, food or exercise. For example, by monitoring D-amino acid levels and blood glucose level in vivo, it is possible to assist in diagnosing and evaluating carbohydrate metabolism disorder, analyzing drug action mechanisms, screening for effects and toxicity, selecting treatment methods and drugs, and deciding on doses and dosing periods. Because D-amino acid levels in body fluids are also affected in other diseases such as kidney disease, D-amino acid levels may be used in analysis as values calibrated with kidney function markers such as creatinine or other markers, for the purpose of discriminating such other diseases.

According to the invention, evaluation of carbohydrate metabolism may also employ casual blood glucose level, fasting blood glucose level, blood insulin level, body weight, intra-peritoneal glucose tolerance test (IGTT:) or insulin tolerance test (ITT:).

Thus, the method for modulating carbohydrate metabolism by modulating of D-amino acid levels in vivo is highly useful for prevention, treatment or diagnosis of carbohydrate metabolism disorder.

According to one embodiment, the invention also provides a method for modulating carbohydrate metabolism of a subject, wherein the method includes administering a modulating agent for the amount of D-amino acids in vivo to a subject in need thereof.

According to another embodiment, the invention provides use of a modulating agent for the amount of D-amino acids in vivo, for production of a pharmaceutical composition for modulation of carbohydrate metabolism.

According to yet another embodiment, the invention provides a method for assessing the state of carbohydrate metabolism of a subject by monitoring the level of D-amino acids in a subject in vivo.

According to yet another embodiment, the invention provides a method of screening for drugs or candidates that can modulate D-amino acid levels in vivo, by measuring D-amino acid levels in tissues, cells, organs or body fluids before and after administration of a drug such as an antihypertensive drug or diabetic nephropathy therapeutic agent.

### EXAMPLES

The present invention will now be explained in greater detail by Examples, with the understanding that the invention is not limited in any way by the Examples. The statistical processing used was t test, unless otherwise specified.

### Example 1: Relationship Between Loss of Activity of D-Amino Acid Metabolic Enzymes and Carbohydrate Metabolism and Blood Glucose Level

Knockout (DAO-KO) mice and rats for the D-amino acid oxidase gene have suppressed D-amino acid oxidation (decomposition) and increased neutral and basic D-amino acid levels in vivo (NPL 1, NPLs 10 to 12), knockout (DDO-KO) mice for the D-aspartate oxidase gene likewise have increased acidic amino acid levels in vivo (NPL 13), and knockout (SRR-KO) mice for the serine racemase gene have suppressed D-serine synthesis and reduced D-serine levels in vivo (NPL 1). After removing the left kidney of 7-week-old DAO-KO mice (n=5 to 6), DDO-KO mice (n=3 to 6), SRR-KO mice (n=5), or C57BL/6(B6) mice (n=10) as a control (−2 weeks: −2 w), they were administered streptozotocin (STZ) which is toxic to pancreatic ß cells, at 50 mg/kg (0) weeks) to create D-amino acid- and carbohydrate metabolism-modified mice (type 1 diabetes model), and blood insulin measurement (−1 weeks to 17 weeks), insulin tolerance testing (−1 weeks, 2 weeks, 15 weeks), intra-peritoneal glucose tolerance testing (−1 weeks), casual blood glucose measurement (−1 weeks to 18 weeks), fasting blood glucose measurement (−1 weeks to 17 weeks), insulin resistance index analysis (HOMA-IR) (−1 weeks to 17 weeks) and systolic/diastolic blood pressure measurement (−1 weeks to 18 weeks) were conducted as appropriate.

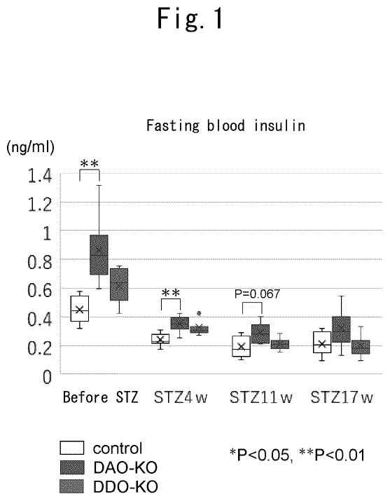

Before STZ administration, the blood insulin levels in the DAO-KO group, and DDO-KO group were higher than the B6 group, indicating increased insulin secretion ability. The blood insulin levels decreased after STZ administration, but higher levels than the B6 group were maintained in the DAO-KO group throughout the period and in the DDO-KO group up to 4-weeks, indicating the ability to suppress insulin secretion disorder (FIG. 1).

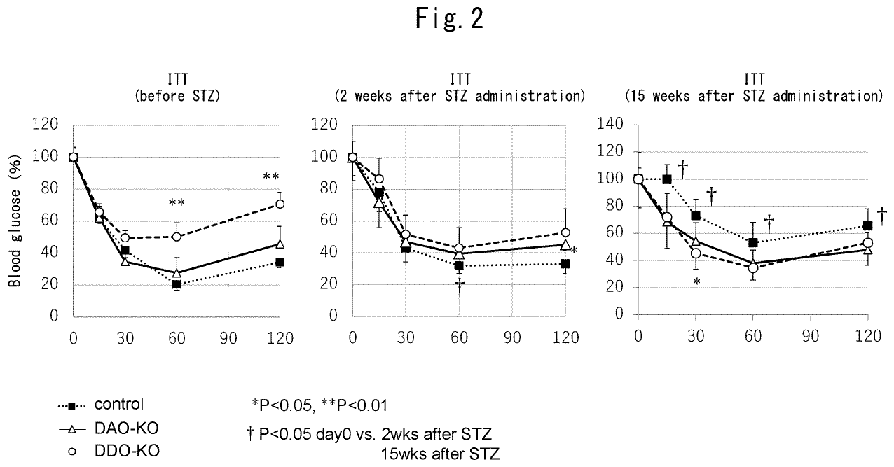

In comparison during the 0 to 15 weeks of insulin tolerance testing (ITT), the blood glucose levels increased and the insulin resistance increased in the B6 group, whereas in the DAO-KO group and DDO-KO group, the blood glucose levels were maintained or lowered, indicating relatively high insulin sensitivity (FIG. 2).

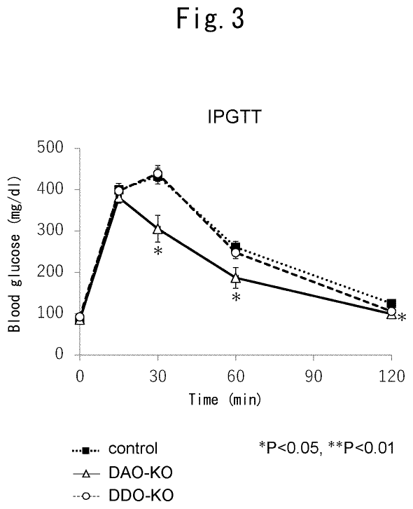

In the intra-peritoneal glucose tolerance testing (IPGTT), the DAO-KO group had a lower value for blood glucose level than the B6 group, indicating improved glucose tolerance. The DDO-KO group showed no difference with the B6 group (FIG. 3).

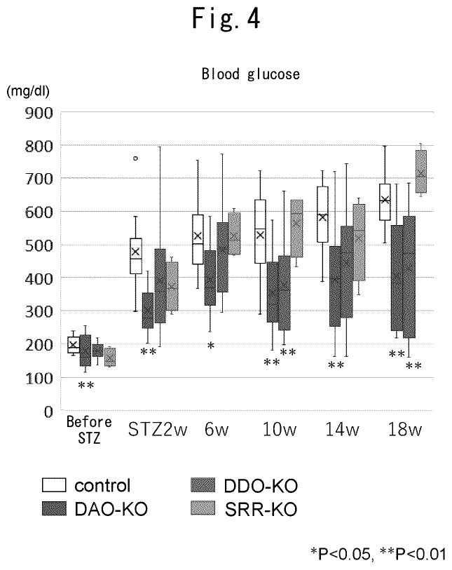

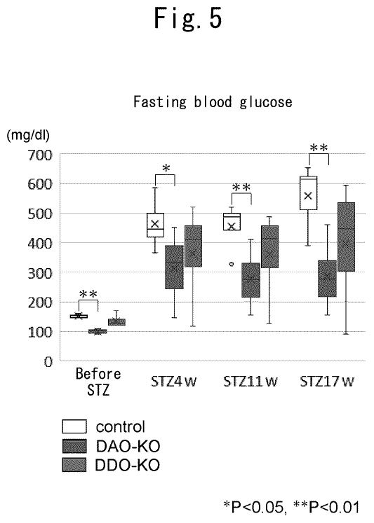

Although the casual blood glucose levels increased after STZ administration, they remained lower in the DAO-KO group and DDO-KO group compared to the B6 group, while the SRR-KO group increased to a higher value (FIG. 4). The fasting blood glucose levels increased after STZ administration, but remained lower in the DAO-KO group and DDO-KO group compared to the B6 group (FIG. 5). These data indicated effects whereby increased D-amino acid levels in vivo decrease blood glucose level, while lower D-amino acid levels increase blood glucose level.

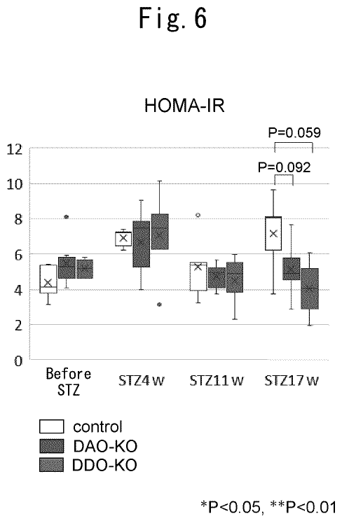

At 17 weeks after STZ administration, the DAO-KO group and DDO-KO group had lower HOMA-IR than the B6 group, indicating that increased insulin resistance had been suppressed. (HOMA-IR=Fasting blood glucose (mg/dl)×insulin concentration (uU/mL)/405) (FIG. 6)

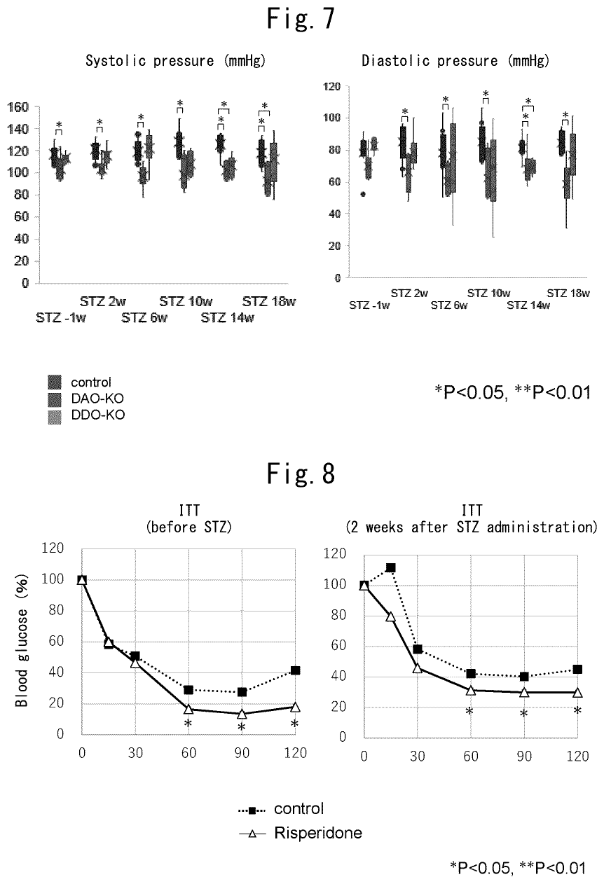

Compared to the B6 group (control group), systolic pressure and diastolic pressure were maintained at lower levels throughout the entire period in the DAO-KO group and from 14 to 18 weeks after STZ administration in the DDO-KO group (FIG. 7).

These results indicated that increase or decrease in D-amino acids in vivo due to loss of activity of a specific D-amino acid metabolic enzyme modulated carbohydrate metabolism, as represented by insulin secretion, insulin resistance and glucose tolerance, and also decreased blood pressure.

### Example 2: Relationship Between D-Amino Acid Metabolic Enzyme Inhibition and Carbohydrate Metabolism and Blood Glucose Level

After removing the left kidney of C57BL/6(B6) mice (−2 weeks: −2 w), a group administered Risperidone aqueous solution as a DAO inhibitor in drinking water (n=3 to 7) and a group administered water as drinking water (n=9 to 11) as a control were administered streptozotocin (STZ) at 50 mg/kg (0) weeks) to create carbohydrate metabolism-modified model mice, and insulin tolerance testing (−1 weeks, 2 weeks), intra-peritoneal glucose tolerance testing (−1 weeks), casual blood glucose measurement (−1 weeks, 2 weeks, 6 weeks) and fasting blood glucose measurement (−1 weeks, 2 weeks) were conducted as appropriate.

In insulin tolerance testing (ITT) before and at 2 weeks after STZ administration, the Risperidone group had lower blood glucose levels compared to the control group, indicating suppressed insulin resistance (FIG. 8).

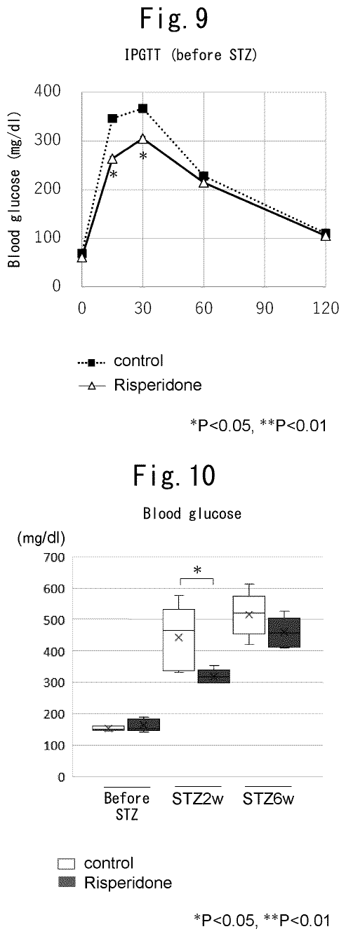

In the intra-peritoneal glucose tolerance testing (IPGTT), the Risperidone group had lower values for blood glucose level than the control group, indicating improved glucose tolerance. (FIG. 9).

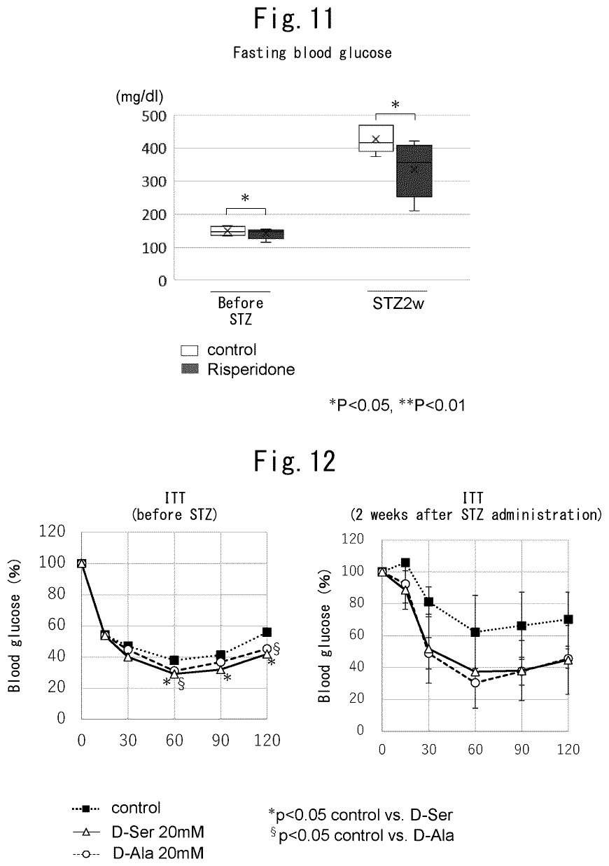

The casual blood glucose levels increased after STZ administration but remained at low levels in the Risperidone group compared to the control group (FIG. 10). The fasting blood glucose levels also increased after STZ administration but remained at low levels in the Risperidone group compared to the control. These data indicated that inhibition of oxidation (decomposition) of D-amino acids in vivo by a DAO inhibitor can lower blood glucose level (FIG. 11).

The results indicated that increased or decrease in D-amino acids in vivo due to inhibition of a specific D-amino acid metabolic enzyme modulated carbohydrate metabolism, as represented by insulin resistance and glucose tolerance.

### Example 3: Relationship Between D-Amino Acid Administration and Carbohydrate Metabolism and Blood Glucose Level

After removing the left kidney of C57BL/6(B6) mice (−2 weeks: −2 w), a group administered an aqueous solution of 20 mM D-alanine (n=5 to 8), 80 mM D-alanine (n=3 to 4), 20 mM D-serine (n=6 to 7) or 80 mM D-serine (n=4) as drinking water, and a group administered water as drinking water as a control (n=8) were administered streptozotocin (STZ) at 50 mg/kg (0 weeks) to create carbohydrate metabolism-modified model mice, and insulin tolerance testing (−1 weeks, 2 weeks), casual blood glucose measurement (0 weeks to 18 weeks), fasting blood glucose measurement (0 weeks to 16 weeks) and systolic/diastolic blood pressure measurement (−1 weeks to 18 weeks) were conducted as appropriate.

In insulin tolerance testing (ITT) before and at 2 weeks after STZ administration, the 20 mM D-alanine administered group and the 20 mM D-serine administered group had lower blood glucose levels and lower insulin resistance compared to the control group, indicating higher insulin sensitivity (FIG. 12).

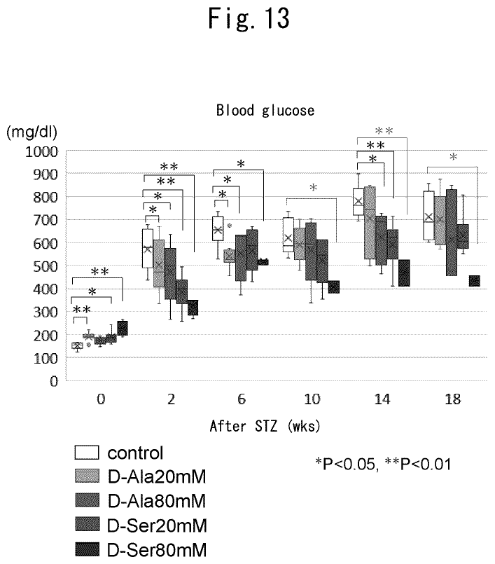

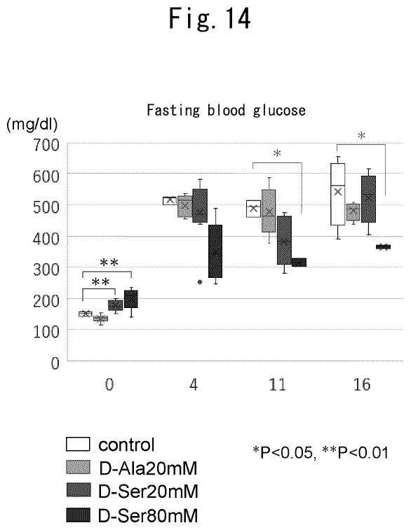

The casual blood glucose levels increased after STZ administration but remained at a low levels in the D-alanine administered group and D-serine administered group in a dose-dependent manner compared to the control group (FIG. 13). The fasting blood glucose levels increased after STZ administration but remained at a low levels in the D-alanine administered group and D-serine administered group in a dose-dependent manner compared to the control group (FIG. 14).

These data indicated that D-amino acid administration decreases blood glucose levels.

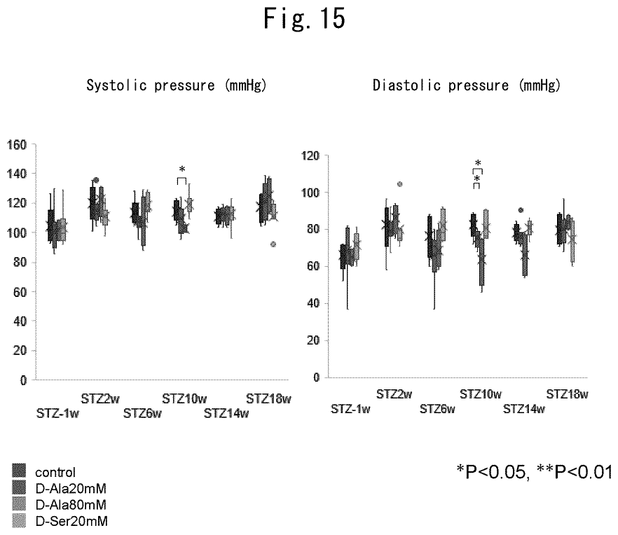

At 10 weeks after STZ administration, systolic pressure and diastolic pressure were lowered in the 80 mM D-alanine administered group compared to the B6 group (FIG. 15).

These results indicated that increase in D-amino acid levels in vivo by D-amino acid administration modulated carbohydrate metabolism as represented by insulin resistance, while also lowering blood pressure.

Since one individual died within 18 weeks in the 80 mM D-serine administered group, dosage setting is essential in light of D-serine toxicity.

### Example 4: Relationship Between D-Amino Acid-Eliminated Diet and Carbohydrate Metabolism and Blood Glucose Level

After removing the left kidney of C57BL/6(B6) mice (−2 weeks: −2 w), a D-amino acid-eliminated diet (D-amino acid-free diet, D-fd) was provided, and groups administered aqueous solution of 20 mM D-alanine (n=5), 20 mM D-serine (n=5), 20 mM D-proline (n=5), 20 mM D-leucine (n=5) or water (n=6 to 14) in drinking water, and a group given normal diet and water as a control (n=5), were administered streptozotocin (STZ) at 50 mg/kg (0 weeks) to create carbohydrate metabolism-modified model mice, and blood insulin measurement (−1 weeks, 3 weeks), casual blood glucose measurement (−1 weeks, 2 weeks), fasting blood glucose measurement (−1 weeks, 3 weeks) and measurement of insulin and glucagon in pancreatic islets of Langerhans by antibody staining, were conducted as appropriate.

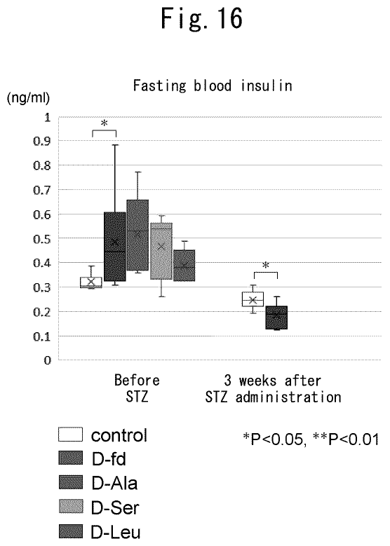

The blood insulin levels decreased after STZ administration, but the D-amino acid-eliminated diet group (D-amino acid free diet: D-fd) had lower values compared to the control group, indicating advanced insulin secretion disorder. (FIG. 16).

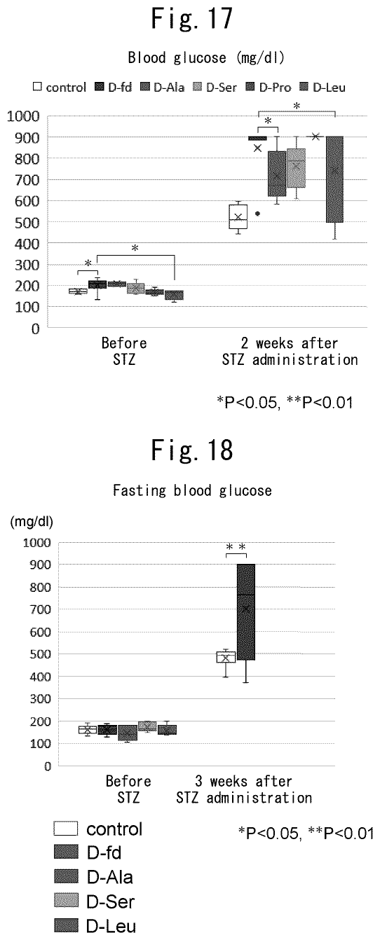

The casual blood glucose levels increased after STZ administration, with a higher level in the D-amino acid-eliminated diet group compared to the control group, but increase in casual blood glucose level was suppressed in the D-alanine administered group, the D-serine-administered group and the D-leucine-administered group (FIG. 17). The fasting blood glucose levels increased after STZ administration, with a higher level in the D-amino acid-eliminated diet compared to the control. These data indicated that decrease in D-amino acid levels in vivo by a D-amino acid-eliminated diet increases blood glucose level, and can be suppressed by increase in D-amino acid levels in vivo by D-amino acid administration (FIG. 18).

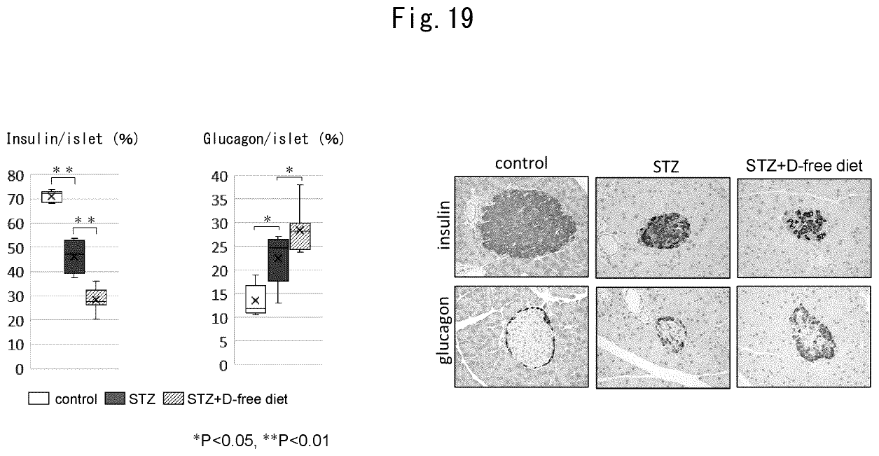

Following STZ administration, the insulin level in the pancreatic islets of Langerhans in the D-amino acid-eliminated diet group decreased, and the glucagon level increased, compared to the control group (FIG. 19).

These results indicated that decrease in D-amino acid levels in vivo by a D-amino acid-eliminated diet modulated carbohydrate metabolism, as represented by insulin secretion into the blood and insulin and glucagon levels in the pancreatic islets of Langerhans.

### Example 5: Relationship Between Non-Insulin-Dependent Diabetes Model and Carbohydrate Metabolism and Blood Glucose Level

Casual blood glucose levels were periodically (5 to 18 weeks) measured using 5- to 8-week-old mice in a group of db/db mice (type 2 diabetes model) exhibiting overeating/obesity due to leptin receptor disorder, administered drinking water with 20 mM D-alanine (n=12), a D-amino acid oxidase gene knockout (DAO-KO) group (n=5) and a control group (n=8).

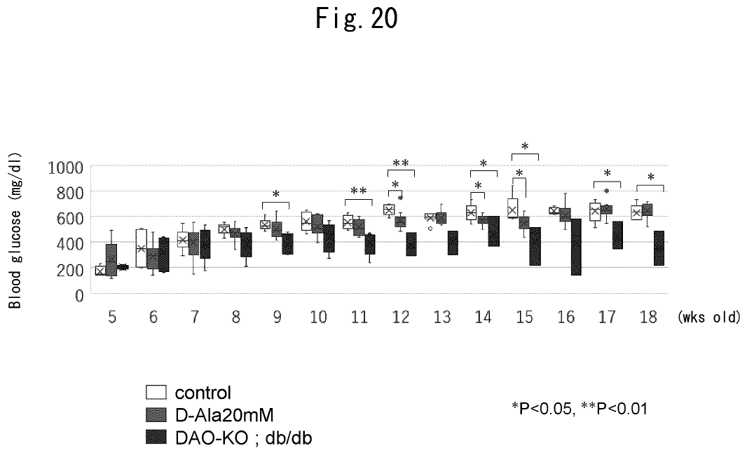

The casual blood glucose levels were at lower levels in the D-alanine-administered group and DAO-KO group compared to the control group, with the DAO-KO group exhibiting the lower level (FIG. 20).

These results indicated that increased D-amino acids in vivo by administration of D-amino acids and loss of activity of a specific D-amino acid metabolic enzyme modulated carbohydrate metabolism, as represented by blood glucose levels.

1.-28. (canceled)

29. A method for modulating carbohydrate metabolism and/or blood pressure of a subject, wherein the method comprises:
administering a modulating agent for the amount of a D-amino acid in vivo to the subject in need thereof.

30. (canceled)

31. The method according to claim 29, wherein the modulating of blood pressure is lowering of blood pressure.

32. The method according to claim 29, wherein the modulating of carbohydrate metabolism is modulating of the glucose level in blood.

33. The method according to claim 29, wherein the modulating agent is a D-amino acid, or modified form or derivative thereof.

34. The method according to claim 33, wherein the D-amino acid is selected from the group consisting of D-alanine, D-serine and D-leucine.

35. The method according to claim 29, wherein the modulating of carbohydrate metabolism is improvement in carbohydrate metabolism.

36. The method according to claim 29, wherein the modulating agent is an agent that modulates the activity of a protein associated with absorption, transport, distribution, metabolism or excretion of a D-amino acid.

37. The method according to claim 36, wherein the metabolism is decomposition and/or synthesis.

38. The method according to claim 36, wherein the agent that modulates the activity of the protein is a modulating agent for gene expression of the protein.

39. The method according to claim 36, wherein the modulating agent is selected from the group consisting of antisense RNA or DNA molecules, RNAi-inducible nucleic acids, microRNA (miRNA), ribozymes, genome editing nucleic acids and their expression vectors, low molecular compounds, aptamers, antibodies, antibody fragments, and combinations of the foregoing.

40. The method according to claim 36, wherein the protein is selected from the group consisting of D-amino acid oxidase, D-aspartate oxidase and serine isomerase.

41. The method according to claim 29, wherein the modulating agent is Risperidone.

42. The method according to claim 29, wherein the carbohydrate metabolism is carbohydrate metabolism by secretion and/or action of insulin and/or glucagon.

43. The method according to claim 29, wherein the carbohydrate metabolism is carbohydrate metabolism represented by insulin resistance or sensitivity, or glucose tolerance.

44. The method according to claim 29, wherein the source of the D-amino acid is symbiotic bacteria.

45. The method according to claim 29, wherein the subject is a subject with a carbohydrate metabolism disorder.

46. The method according to claim 45, wherein the carbohydrate metabolism disorder is diabetes.

47. The method according to claim 29, wherein the subject is a subject with a diabetic complication.

48. The method according to claim 47, wherein the diabetic complication is selected from the group consisting of retinopathy, nephropathy, neuropathy and arteriosclerosis.

49. A method for modulating blood pressure of a subject, wherein the method comprises:
administering a modulating agent for the amount of a D-amino acid in vivo to the subject in need thereof.
